(19) United States
(12) Patent Application Publication
(10) Pub. No.: US 2008/0103254 A1
(43) Pub. Date: May 1, 2008

(54) ACTIVE ENERGY BEAM-CURABLE INK

(75) Inventor: Keisuke Asada, Shibata-gun (JP)

Correspondence Address: OBLON, SPIVAK, MCCLELLAND MAIER & NEUSTADT, P.C., 1940 DUKE STREET, ALEXANDRIA, VA, 22314, US

(73) Assignee: Tohoku Ricoh Co., Ltd., Shibata-gun (JP)

(21) Appl. No.: 11/850,459

(22) Filed: Sep. 5, 2007

(30) Foreign Application Priority Data
Oct. 31, 2006 (JP) 2006-295946
Jun. 6, 2007 (JP) 2007-151004

(51) Int. Cl. C08G 63/00; C07C 69/00

(52) U.S. Cl. 524/832; 560/129

(57) ABSTRACT

To provide an active energy beam-curable ink which includes active energy beam-curable monomer components, wherein at least one of the active energy beam-curable monomer components contains a unit represented by the following structural formula (1) in its molecule.
—(CO—CH2CH2CH2CH2CH2—O)— Structural formula (1)

### BACKGROUND OF THE INVENTION

1. Field of the Invention

The present invention relates to an active energy beam-curable ink that has an excellent curing property and in which the molecular weight of monomer components which are associated with safety issues can be increased without increasing the viscosity of the ink.

2. Description of the Related Art

Conventionally, emulsion inks have been used in screen printing, where a stencil having holes punched by thermal digital platemaking is used and images are formed from ink passing through the holes of the stencil. However, the emulsion inks are slow to dry, and thus, when sheets of paper covered with a large portion of solid images are printed and when they are placed one upon another, the emulsion inks cause occurring of offsets, or transferring of ink from neighboring sheets, resulting in causing smears on the printed sheets.

In view of the foregoing, active energy beam-curable inks have been replacing the conventional emulsion inks. The active energy beam-curable inks are cured instantly with ultraviolet irradiation. Thus, the active energy beam-curable inks are advantageous in, as they dry faster than commonly used W/O (water-in-oil) type emulsion inks, preventing occurring of offsets.

For example, Japanese Patent Application Laid-Open (JP-A) No. 2000-290572 discloses an active energy beam-curable ink that contains a carbon black as a colorant. It suggests that, in the active energy beam-curable ink, excellent concentration of printed images and excellent storage stability of the ink can be achieved by adjusting the coloring ability and volatility of the carbon black to be in a predetermined range. It also suggests using, for example, di-pentaerythritol hexaacrylate (DPHA) as a polymerized polyfunctional component to achieve the above-stated objects.

However, DPHA has a high viscosity which ranges from 4,000 mPa·s to 8,000 mPa·s at 25° C., and thus the formulating amount of DPHA is limited to a certain level when the active energy beam-curable ink needs to have a low viscosity. For this reason, the disclosed technique is disadvantageous in obtaining fast curing speed in the disclosed ink. The disclosed technique also uses acrylate modified phenol ethylene oxide as a polymerizable monomer. Acrylate modified phenol ethylene oxide is hazardous to the skin, as it has a relatively small molecular weight which ranges from 236 to 324 and a low viscosity which ranges from 10 mPa·s to 40 mPa·s at 25° C.

Moreover, acrylic monomers that are commonly used in active energy beam-curable inks have a high reactivity, and thus the skin is often irritable to such inks, indicating potential danger of using the inks. Thus, monomers having lower skin irritability have been used in recent years. In particular, monomers having a primary irritation index (PII) of 2.0 or lower have been commonly used for inks to obtain low skin irritability therein.

In such ink, lower skin irritability is generally achieved by adding ethyleneoxide and/or propyleneoxide to molecules in the ink to increase its molecular weight; however, the viscosity of the ink usually increases with increasing molecular weight.

For the above-stated reasons, there is a demand for an active energy beam-curable ink that has an excellent curing property and in which the molecular weight of monomer components which are associated with safety issues can be increased without increasing the viscosity of the ink.

### BRIEF SUMMARY OF THE INVENTION

An object of the present invention is to solve the forementioned problems and to achieve the following objects. That is, to provide an active energy beam-curable ink that has an excellent curing property and in which the molecular weight of monomer components which are related to safety issues can be increased without increasing the viscosity of the ink.

The inventor of the present invention conducted studies in order to solve the forementioned problems and established that, by using monomer components modified with ε-caprolactone, it is possible to obtain an active energy beam-curable ink that has an excellent curing property and in which the molecular weight of the monomer components which are related to safety issues can be increased without increasing the viscosity of the ink. Although the reason why using the monomer components modified with ε-caprolactone can provide the active energy beam-curable ink having such characteristics is yet to discovered, it is considered that modification of the components with ε-caprolactone lowers the specific gravity thereof, and thus steric effects of functional groups having ε-caprolactone have more influence to lower the viscosity than to increase the molecular weight.

Additionally, in terms of safety, the active energy beam-curable ink of the present invention is considered to have low skin irritability, as larger molecular structures expanded by modification with r-caprolactone of the components prevent the component from penetrating the skin.

The present invention is based on the above-stated knowledge of the inventor, and means to solve the problems are as follows:

<1>. An active energy beam-curable ink which includes active energy beam-curable monomer components, wherein at least one of the active energy beam-curable monomer components contains a unit represented by the following structural formula (1) in its molecule.
—(CO—CH2CH2CH2CH2CH2—O)— Structural formula (1)

<2>. The active energy beam-curable ink according to <1>, wherein at least one active energy beam-curable monomer component having the unit represented by the structural formula (1) in its molecule is di-pentaerythritol hexaacrylate modified with ε-caprolactone and is represented by the following structural formula (2).

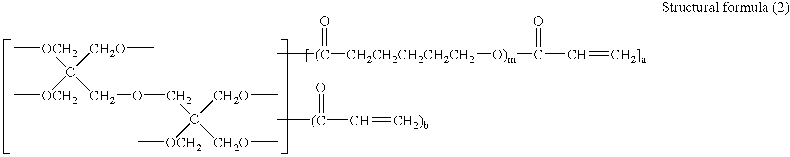

Where “a”, “b” and “m” are average values, “a” is an integer in the range of 1 to 6, “b” is an integer in the range of 0 to 6, a+b=6, and “m” is an integer in the range of 1 to 2.

<3>. The active energy beam-curable ink according to <2>, wherein “a” represents 6 and “b” represents 0 in the structural formula (2).

<4>. The active energy beam-curable ink according to <1>, wherein at least one active energy beam-curable monomer component having the unit represented by the structural formula (1) in its molecule is hydroxypivalate neopentylglycol ester diacrylate modified with ε-caprolactone and is represented by the following structural formula (3).
CH₂═(CHCO—(OCH₂CH₂CH₂CH₂CH₂CO)ₘ—OCH₂C(CH₃)₂—COO—OH₂—C(CH₃)₂—CH₂—O—(COCH₂CH₂CH₂CH₂CH₂O)ₙ—COCH═CH₂ Structural formula (3)

Where “a”, “b” and “m” are average values, “m” is an integer in the range of 0 to 4, “n” is an integer in the range of 0 to 4, and m+n=2 to 4.

<5>. The active energy beam-curable ink according to <1>, wherein all of the active energy beam-curable monomer components have the unit represented by the structural formula (1) in their molecules.

<6>. The active energy beam-curable ink according to <1>, which is used for screen printing, where a stencil having holes punched by thermal digital platemaking is used and images are formed from ink passing through the holes.

<7>. The active energy beam-curable ink according to <I>, which further includes a monomer having at least an ethyleneoxide (EO) unit represented by the structural formula (4).
—(CH2CH2O)— Structural formula (4)

<8>. The active energy beam-curable ink according to <7>, wherein the number of the ethyleneoxide (EO) units represented by the structural formula (4) is in the range of 6 to 14.

<9>. The active energy beam-curable ink according to <1>, which further includes a silica. According to the present invention, it is possible to provide an active energy beam-curable ink that has an excellent curing property and in which the molecular weight of monomer components which are related to safety issues can be increased without increasing the viscosity of the ink.

### DETAILED DESCRIPTION OF THE INVENTION

The active energy beam-curable ink of the present invention includes active energy beam-curable monomer components, a colorant, a dispersant, a polymerization initiator, and further includes other component(s) such as an extender pigment, a polymerization inhibitor, a vegetable oil, an antioxidant and/or a synergist in accordance with necessity.

The term “active energy beam cure” or “active energy beam curable” as used herein refers to polymerization, initiated by active energy beam irradiation, and cure of the monomer components. The state of the cured monomer components can be confirmed by, for example, touching ink after exposed to active energy beam irradiation. The active energy beam may be, for example, an ultraviolet ray or electron beam.

The ink of the present invention may be made of a material curable by radical polymerization or a material curable by cationic polymerization.

<Active Energy Beam-Curable Monomer Component>

At least one of the active energy beam-curable monomer components has a unit in its molecule. The unit is represented by the following structural formula (1). The unit represented by the structural formula (1) is a ring-opened ε-caprolactone.
—(CO—CH2CH2CH2CH2CH2—O)— Structural Formula (1)
The active energy beam-curable monomer component having the units represented by the structural formula (1) in its molecule is not particularly limited, and it can be appropriately selected according to the purpose; however, for further increasing the molecular weight of the monomer component without increasing the viscosity of the ink, at least one of the monomer components is preferably di-pentaerythritol hexaacrylate modified with ε-caprolactone, preferably represented by the following structural formula (2).

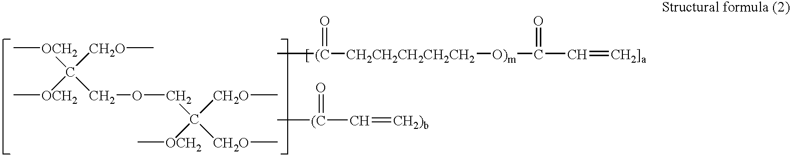

Where “a”, “b” and “m” are average values, “a” is an integer in the range of 1 to 6, “b” is an integer in the range of 0 to 6, and a+b=6. And “m” is an integer in the range of 1 to 2.

More specifically, the monomer component represented by the structural formula (2) is preferably a compound in which the average values m, a and b are one of the following combinations: m=1, a=2 and b=4; m=1, a=3 and b=3; m=r, a=6 and b=0; and m=2, a=6 and b=0. Particularly, the monomer component is more preferably a compound in which a=6 and b=0 for increasing the molecular weight of the monomer component without increasing its viscosity.

The monomer component represented by the structural formula (2) may be selected from commercial available products such as KAYARAD series (DPCA-20, DPCA-30, DPCA-60 and DPCA-120) manufactured by Nippon Kayaku Co., Ltd.

The added amount of the monomer component represented by the structural formula (2) is not particularly limited and can be set at a suitable level according to the purpose. For example, when the monomer component is used in an ink, the added amount is preferably in the range of 10% by mass to 97% by mass, and more preferably in the range of 30% by mass to 85% by mass. When the monomer component is used as an overcoating agent, the added amount is preferably in the range of 5% by mass to 99% by mass, and more preferably in the range of 20% by mass to 95% by mass.

The active energy beam-curable monomer component having the units represented by the structural formula (1) in its molecule is preferably hydroxypivalate neopentylglycol ester diacrylate modified with ε-caprolactone. The compound represented by the following structural formula (3) is also preferable.
CH₂═(CHCO—(OCH₂CH₂CH₂CH₂CH₂CO)ₘ—OCH₂C(CH₃)₂—COO—OH₂—C(CH₃)₂—CH₂—O—(COCH₂CH₂CH₂CH₂CH₂O)ₙ—COCH═CH₂ Structural formula (3)

Where “a”, “b” and “m” are average values, “m” is an integer in the range of 0 to 4, “n” is an integer in the range of 0 to 4, and m+n=2 to 4.

More specifically, the monomer component represented by the structural formula (3) is suitably a compound in which m+n=2 or 4 in the structural formula. Of these compounds, the one in which m+n=4 is more preferable from the viewpoint of safety.

The monomer component represented by the structural formula (3) may be selected from commercial available products such as KAYARAD series (HX-220 and HX-620) manufactured by Nippon Kayaku Co., Ltd.

The viscosity of the monomer component represented by the structural formula (3) is preferably 2,000 mPa·s or lower, more preferably 800 mPa·s or lower and further preferably 400 mPa·s or lower at 25° C. for minimizing the change in the viscosity against the change of temperature. In this regard, however, the viscosity of the monomer component is preferably 20 mPa·s or higher and more preferably 100 mPa·s or higher for preventing image defects caused by excessive flowability of ink.

The added amount of the monomer component represented by the structural formula (3) is not particularly limited and can be set at a suitable level according to the purpose. For example, when the monomer component is used in an ink, the added amount is preferably in the range of 10% by mass to 97% by mass, and more preferably in the range of 30% by mass to 85% by mass. When the monomer component is used in an overcoating agent, the added amount is preferably in the range of 5% by mass to 99% by mass, and more preferably in the range of 20% by mass to 95% by mass.

Only either one of the compounds, represented by the structural formula (2) and (3), may be added into ink, while adding both compounds is more preferable for further improving the curing property of ink.

Examples of monomer components having the unit represented by the structural formula (1) include caprolactone modified trimethylolpropane triacrylate, pentaerythritol tetracaprolactate tetraacrylate, ditrimethylolpropane tetracaprolactate tetraacrylate, ε-caprolactone modified tris(acryloxyethyl) isocyanurate, ω-carboxy-polycaprolactone (n=2) monoacrylate, and caprolactoneacrylate

Specific structures of the active energy beam-curable monomer components having the unit represented by the structural formula (1) in the above-stated molecule, as well as the existence of the monomer components in ink, can be analyzed in terms of their structural formula and relative proportions. They can be obtained by analyzing monomer components extracted from obtained ink using, for example, centrifugal separation or Soxley extraction and concentrated after the extraction. The monomer components can be analyzed by an instrumental analysis such as gas chromatography, liquid chromatography, FT-IR (Fourier transform infrared spectrophotometer), NMR (nuclear magnetic resonance apparatus), an ultimate analysis and a mass analysis.

—Other Monomer Components—

Other monomer components which contain no unit represented by the structural formula (1) in their molecules may be contained in the ink as active energy beam-curable monomer components.

Examples of the other monomer components include acrylic acid-modified monomers and oligomers including urethanes, epoxies, polyesters and polyols. The term “acrylic acid” refers to both acrylic acid and methacrylic acid.

Examples of the above-stated monomers include monofunctional and polyfunctional acrylate monomers. Examples of the acrylate monomer include: dicyclo-pentell ethylacrylate; isobonyl acrylate; acrylate modified phenol ethylene oxide; tripropylene glycol diacrylate; hydroxypivalic acid neopentylglycol ester; 1,6-hexanediol diacrylate; bisphenol A diglycidyl ether diacrylate; triethylene glycol diacrylate; tetraethylene glycol diacrylate; polyethylene glycol diacrylate (where the number of ethylene oxide units is in the range of 5 to 14); trimethylolpropane triacrylate; pentaerythritol triacrylate; propylene oxide modified glycerol triacrylate; ethylene oxide (EO) modified trimethylolpropane triacrylate (where EO is in the range of 1 to 20); propylene oxide (PO) modified trimethylolpropane triacrylate (where PO is in the range of 1 to 6); pentaerythrito tetraacrylate; ditrimethylolpropane tetra acrylate; pentaerythritol ethoxy tetra acrylate; dipentaerythritol pentaacrylate; dipentaerythritol hexaacrylate; 1,4-butandiol dimethacrylate; hexanediol dimethacrylate; ethylene glycol dimethacrylate; triethylene glycol dimethacrylate; tetraethylene glycol dimethacrylate; polyethylene glycol dimethacrylate (where the number of ethylene oxide units is in the range of 5 to 14); neopentyl glycol dimethacrylate; and trimethylolpropanetrimethacrylate.

Examples of the oligomer include epoxy acrylates, epoxidized oil acrylates, urethane acrylates, unsaturated polyesters, polyester acrylates and vinyl acrylates.

A monomer used together in the present invention preferably has an ethylene oxide unit. Using a diacrylate or dimethacrylate having the number of the ethylene oxide units in the range of 5 to 14 is more preferable. Using the diacrylate is further preferable from the viewpoint of curing property. The added amount of such monomer is preferably less than 20% by mass based on the total mass of the ink.

The above-stated other monomer components can be used alone or in combination.

The viscosity of the other monomer components is preferably 2,000 mPa·s or lower, more preferably 800 mPa·s or lower and further preferably 400 mPa·s or lower at 25° C. for minimizing the change in the viscosity against the change of temperature. In this regard, however, the viscosity of the other monomer components is preferably 20 mPa·s or higher and more preferably 100 mPa·s or higher for preventing image defects caused by excessive flowability of ink.

The added amount of the other monomer components is not particularly limited and can be set at a suitable level in accordance with the purpose. For example, when the other monomer components are added into ink, the added amount is preferably 85% by mass or less and more preferably 50% by mass or less based on the total mass of the ink. In terms of safety, the ink preferably contains only the monomer component modified with ε-caprolactone and contains none of the other monomer components.

<Colorant>

The above-stated colorant is not particularly limited and can be appropriately selected from insoluble colorants of various colors, including known pigments, dyes and disperse dyes.

Examples of the colorant include carbon blacks such as acetylene black, channel black and furnace black; metal powders such as an aluminum powder and bronze powder; inorganic pigments such as red oxides, yellow lead, ultramarine blue, chromium oxides and titanium oxides; azo pigments such as insoluble azo pigments, azolake pigments and condensed azo pigments; phthalocyanine pigments such as metal-free phthalocyanine pigment and copper phthalocyanine pigment; polycondensed pigments such as anthraquinone dyes, quinacridon dyes, isoindolinone dyes, isoindoline dyes, dioxadin dyes, threne dyes, perylene dyes, perynone dyes, thioindigo dyes, quinophthalone dyes and metallic complexes; organic pigments such as lakes of acid or basic dyes; oil-soluble dyes such as diazo dyes and anthraquinone dyes; and fluorescent pigments.

The above-stated fluorescent pigment is preferably a synthetic resin solid solution type which can be produced by, for example, mass-polymerizing a synthetic resin, dying the resin or dissolving fluorescent dyes of various colors into the resin to obtain a colored mass resin during or after the polymerization, and crushing the thus obtained resin into fine articles. Examples of the synthetic resin into which the dyes are added include melamine resin, urea resin, sulfonamides resin, alkyd resin and polyvinylchloride resin.

The above-stated colorant may be selected from commercial available products. Examples thereof include MA-100, MA-100S, MA-7, MA-70, MA-77, MA-11, #40 and #44 (all manufactured by Mitsubishi Chemical Corporation); Raven1100, Raven1080, Raven1255, Raven760 and Raven410 (all manufactured by Columbia Carbon Company); and MOGUL-L, MOGUL-E and PEARLS-E (all manufactured by CABOT JAPAN K.K.).

Those colorants can be used alone or in combination.

The colorant exists in the ink in a dispersed state. The average particle diameter of the dispersed colorant in the ink is not particularly limited, and can be a suitable measure according to the purpose; it is preferably in the range of 0.1 μm to 10 μm and more preferably 0.1 μm to 1.0 μm. When the average particle diameter is less than 0.1 μm, desired image density may not be obtained as pigments infiltrate into paper immediately after the ink is placed on paper, and when more than 10 μm, stability of the ink may be degraded.

The added amount of the colorant is not particularly limited, and can be set at a suitable level in accordance with the purpose, while, in general, the added amount is preferably in the range of 2% by mass to 15% by mass based on the total mass of the ink.

<Dispersant>

The dispersant is a component with a function of dispersing colorant and dispersing extender pigment which may be added upon necessity.

The dispersants are not particularly limited and can be appropriately selected according to the purpose. They can be selected from, for example, nonionic surfactants such as sorbitan fatty acid esters (such as sorbitansesquioleate), polyglyceryl fatty acid esters (such as hexaglycerin polyricinoleate), polyoxyethylene, glycerin fatty acid ester, polyoxyethylene sorbitan fatty acid ester, polyoxyethylene alkyl ether, polyoxyethylene alkylamine and polyoxyethylene fatty acid amide; high molecular compounds of alkylamines; compounds of aluminum chelates; copolymerized high molecular compounds of styrene—maleic anhydrides; high molecular compounds of polycarboxylic acid esters; aliphatic polyvalent carboxylic acids; amine salts of high molecular polyesters; ester anionic surfactants; long-chain amine salts of high molecular weight polycarboxylic acids; salt of long-chain polyamino amides and polyacid polyesters; compounds of polyamides; phosphoric acid ester surfactants; salt of alkyl sulfo-carboxylic acids; sulfonates; α-olefin sulfonates; dioctyl sulfosuccinate salts; polyethyleneimine; alkanolamides salts; and resins, such as alkyd resins, having function of dispersing insoluble colorants.

The dispersants may be selected from commercial available products such as SOLUSPHASE series (including S3000, S5000, S9000, S13240, S13940, S16000, S17000, S20000, S24000, S26000, S27000, S28000, S31845, S31850, S32000, S32550, S33000, S34750, S36000, S39000, S41090 and S53095) manufactured by Lubrizol Japan Ltd.,; PLANE-ACT AL-M and AJISPER series (including PB711, PM821, PB821, PB811, PN411 and PA111) manufactured by Ajinomoto Fine-Techno Co., Inc.; and 6220, 6225, 6230 and 5244 manufactured by EFKA Additives.

Those dispersants can be used alone or in combination.

The added amount of the dispersant is not particularly limited, and it can be set at a suitable level in accordance with the purpose, while the added amount is preferably 40% by mass or less and more preferably in the range of 2% by mass to 35% by mass based on the total mass of contained colorants and extender pigments.

<Polymerization Initiator>

The polymerization initiators are not particularly limited and can be appropriately selected according to the purpose. Examples of the polymerization initiators include radical polymerization initiators such as photoclearable initiators and proton abstracting initiators. Examples of such initiators include benzophenone, acetophenone, 4,4′-bisdiethylamino benzophenone, benzoin and benzoin ethyl ether.

The polymerization initiators may be selected from commercial available products such as an IRGACURE series (2959, 651, 127, 184, 907, 369 and 379) and DAROCUR series (1173 and TPO) manufactured by Ciba Specialty Chemicals K.K.; and KAYACURE DETX-S and KAYACURE-ITX manufactured by Nippon Kayaku Co., Ltd.

Those polymerization initiators can be used alone or in combination depending on light sources.

The polymerization initiator may be used in combination with a sensitizer or a polymerization accelerator. Examples of such agents include aliphatic amines and aromatic amines such as n-butylamine, triethylamine and p-dimethylamine ethyl benzoate. More specific examples of such agents include DAROCUREDB and DAROCUREHA (manufactured by Ciba Specialty Chemicals K.K.); and KAYACUREEPA and KAYACUREDMBI (manufactured by Nippon Kayaku Co., Ltd.).

The contents of the polymerization initiator, sensitizer and polymerization accelerator are not particularly limited and can be set at suitable levels according to the purpose, while the content of each of these agents is preferably in the range of 1% by mass to 25% by mass and more preferably 1% by mass to 10% by mass based on the total mass of the ink.

—Other Components—

The other components in the ink of the present invention are not particularly limited and can be appropriately selected according to the purpose, provided that the effectiveness of the ink of the present invention is not degraded by adding the other components. They may be selected from, for example, extender pigments, polymerization initiators, polymerization inhibitors, vegetable oils, antioxidants and synergists.

—Extender Pigment—

Examples of the extender pigments are not particularly limited and can be appropriately selected according to the purpose. Examples thereof include inorganic particulates such as china clays, silicas, talcs, clays, calcium carbonates, organic clays, barium sulfates, titanium oxides, alumina whites, diatom soil, kaolin, mica and aluminum hydroxides; organic particulates such as polyacrylate esters, polyurethanes, polyesters, polyethylenes, polypropylenes, polyvinylchloride, polyvinylidene chloride, polystyrenes, poly siloxane, phenolic resins and epoxy resins; and fine particles of copolymers of these compounds.

The extender pigments may be selected from commercial available products. Examples of such products include AEROSIL series (including 50, 90G, 130, 200, 300, 380, TT600, COK84 and R972) manufactured by NIPPON AEROSIL CO., LTD.; HAKUENKA TDD and HAKUENKA 0 manufactured by Shiraishi Kogyo Kaisha, Ltd.; TIXOGEL series (including VP, DS, GB, VG, EZ-100, MP-100, MP-200, MPI and MPG) and OPTICEL manufactured by SUD-CHEMIE CATALYSTS JAPAN, INC.; Garamite series (1958, 1210, 2578, ClaytoneGR, ClaytoneHT, and ClaytonePS3) manufactured by Southern Clay Products Corporation; and SG2000 manufactured by NIPPON TALC Co., Ltd.)

Hydrophilic silicas having primary particle diameters of 100 nm or less should preferably used.

The above-stated dispersant is preferably used for the extender pigment. Those extender pigments can be used alone or in combination.

The content of the extender pigment is not particularly limited and can be set at a suitable level according to the purpose, while it is preferably in the range of 0.1% by mass to 50% by mass, more preferably 1% by mass to 15% by mass, and further preferably 2% by mass to 5% by mass based on the total mass of the ink.

—Polymerization Inhibitor—

A polymerization inhibitor may be used in the ink of the present invention for storing the ink more safely and preventing gelation caused by dark reactions.

The polymerization inhibitor is not particularly limited and can be appropriately selected according to the purpose. It can be selected from, for example, hydroquinone, p-benzoquinone, t-butylhydroquinone and p-methoxyphenol (MEHQ).

In most cases, the added amount of the polymerization inhibitors is preferably in the range of 100 ppm to 5,000 ppm and more preferably in the range of 100 ppm to 500 ppm based on the total mass of the ink.

—Vegetable Oil—

One or more vegetable oils may be used for the ink of the present invention in accordance with necessity, provided that the curing property of the ink is not degraded by adding the oils.

The vegetable oil is not particularly limited and can be appropriately selected according to the purpose. It can be selected from, for example, bean oil, rapeseed oil, corn oil, sesame oil, tall oil, cotton seed cake oil, sunflower oil, safflower oil, walnut oil, poppy oil and linseed oil.

Additionally, an esterified vegetable oil may be used as the vegetable oil. Examples of the esters include methylesters, butylesters, isopropylesters and propylesters. Of those above-stated oils, vegetable oils having an iodine value of 100 or more, which are generally so-called drying oils or semidrying oils, are preferable for obtaining better drying characteristic of the ink after it is placed on paper. In this regard, however, vegetable oils having an iodine value of less than 100 may be used when the ink is kept in a printer for a long period of time and it results in occurrences of ink adhesion/fixation.

Those vegetable oils can be used alone or in combination.

When a drying oil and/or semidrying oil having a high iodine value are used as the vegetable oil, they tend to react with oxygen existing in atmosphere, causing dryness and solidification, and, in particular, resulting in solidification of ink in which they are contained. The solidification of the ink then causes clogging of a screen and a reduction in printing speed/image quality. Thus, when such vegetable oil having a high iodine value (or having many unsaturated bonds) is used, it is preferable that the below-mentioned antioxidant be contained in ink for preventing oxidization of fatty acids (such as linolenic acid, linoleic acid and oleic acid) of the vegetable oil.

The added amount of the vegetable oil is not particularly limited and can be set at a suitable level according to the purpose, while it is preferably in the range of 5% by mass to 70% by mass, and more preferably 30% by mass to 50% by mass based on the total mass of the ink.

—Antioxidant—

The above-stated antioxidant is not particularly limited and can be appropriately selected from known compounds according to the purpose. Examples thereof include amine compounds such as diphenyl-phenylenediamine and isopropylphenyl-phenylenediamine; phenolic compounds such as tocopherol and dibutylmethylphenol; and sulfur compounds such as mercaptomethyl-benzimidazole. Those compounds can be used alone or in combination.

The added amount of the antioxidant is not particularly limited and can be set at a suitable level according to the purpose, while it is preferably 10% by mass or less, and more preferably 1% by mass or less based on the total mass of vegetable oil.

Sufficient antioxidant effect may not be obtained when antioxidant is added in a little amount relative to the content of vegetable oil; on the other hand, when it is added in an excessive amount relative to the content of vegetable oil at a time, it may function as a pro-oxidant agent. Thus, the below-mentioned synergist should preferably added for obtaining desired antioxidant effect for vegetable oil with a small amount of antioxidant.

—Synergist—

The synergist itself provides almost no protection against oxidation, but enhances antioxidant effect when it is used in combination with an antioxidant. The synergist is generally acid and is a polyfunctional compound having one or more hydroxyl groups or carboxyl groups.

The above-stated synergist is not particularly limited and can be appropriately selected from known compounds according to the purpose. Examples thereof include methionine, ascorbic acid, threonine, leucin, hydrolyzed milk proteins, vorvaline, ascorbyl palmitate, phenylalanine, cysteine, tryptophan, proline, alanine, glutaminic acid, valine, pepsin digestive fluid of pancreatic proteins, asparagine, arginine, barbiturates, asphenamine, ninhydrin, propanidine, histidine, norleucine, glycerophosphoric acid, liquid of casein hydrolyzed by trypsin, and liquid of casein hydrolyzed with hydrochloric acid. Those compounds can be used alone or in combination.

The added amount of the synergist is not particularly limited and can be set at a suitable level in accordance with the purpose, while the added amount is preferably in the range of 50% by mass to 150% by mass based on the total mass of antioxidant.

<Production Method for Active Energy Beam-Curable Ink>

The production method for the active energy beam-curable ink of the present invention is not particularly limited and can be appropriately selected from known methods according to the purpose. For example, it can be obtained by a dispersion treatment in which necessary components are mixed by a usual process, and then they are dispersed using a dispersion machine such as a three-roll mill.

When the ink is used in screen printing, the viscosity of the ink can be adjusted at a suitable level by changing agitation conditions during the dispersion treatment. The viscosity of the ink is not particularly limited, provided it is suitable to be used in screen printing and a system therefor. The viscosity is preferably in the range of 2 Pa·s to 40 Pa·s, and more preferably in the range of 10 Pa·s to 30 Pa·s when the share rate is 20/s. Additionally, an approximate value of the plastic viscosity of the ink, obtained by the following Casson's equation, is preferably less than 2.0 Pa·s, and more preferably in the range of 0.1Pa·s to 1.0 Pa·s for preventing curling of printed paper:
\sqrt{τ}−\sqrt{τ₀}=\sqrt{Eta×D} Casson's Approximation Formula
where τ represents a shear stress, τ0 represents a yield value, Eta represents a plastic viscosity and D represents a shear velocity.

<Application>

As described above, the active energy beam-curable ink of the present invention has an excellent curing property and in which the molecular weight of monomer components which are related to safety issues can be increased without increasing the viscosity of the ink. Thus, the active energy beam-curable ink of the present invention can be suitably used for screen printing using a rotary screen printer.

### EXAMPLES

Hereinafter, with referring to Examples and Comparative Examples, the invention will be explained in detail; however, the following Examples and Comparative Examples should not be construed as limiting the scope of this invention.

### Examples 1 to 18 and Comparative Examples 1 to 3

### —Preparation of Ultraviolet Ray-Curable Ink for Screen Printing—

Ultraviolet ray-curable inks for screen printing were prepared for Examples 1 to 18 and Comparative Examples 1 to 3, in accordance with the formulations shown in Tables 1 to 4. In this preparation, colorants, dispersants, extender pigments, monomer components and polymerization initiators for each ultraviolet ray-curable ink were mixed, and then they were dispersed using a three-roll mill (manufactured by INOUE MANUFACTURING CO., LTD.) to thereby obtain the inks.

TABLE 1

| | | Viscosity | Molecular | | | | | | |
|---|---|---|---|---|---|---|---|---|---|
| | | (mPa · s) | weight | Comp. Ex. 1 | Ex. 1 | Ex. 2 | Ex. 3 | Ex. 4 | Ex. 5 |
| Colorant | MOGUL-E | — | — | 7.0 | 7.0 | 7.0 | 7.0 | 7.0 | 7.0 |
| | (CABOT Corporation) | | | | | | | | |
| Dispersant | SOLUSPHASE S33000 | — | — | 0.4 | 0.4 | 0.4 | 0.4 | 0.4 | 0.4 |
| | (Lubrizol Japan Ltd.) | | | | | | | | |
| Extender pigment | Aerosil 200 | — | — | 4.0 | 4.0 | 4.0 | 4.0 | 4.0 | 4.0 |
| | (NIPPON AEROSIL CO., | | | | | | | | |
| | LTD.) | | | | | | | | |
| Monomer component | Monomer A | 7,000 | 578 | 23.0 | | | | | |
| (*) | Monomer B | 2,000 | 807 | | 36.0 | | | | |
| | Monomer C | 1,700 | 921 | | | 36.0 | | | |
| | Monomer D | 1,350 | 1,263 | | | | 39.0 | | 47.0 |
| | Monomer E | 1,500 | 1,947 | | | | | 38.0 | |
| | Monomer F | 150 | 427 | 55.6 | 42.6 | 42.6 | 39.6 | 40.6 | |
| | Monomer G | 110 | 540 | | | | | | 31.6 |
| | Monomer H | 250 | 768 | | | | | | |
| | Monomer I | 610 | 776 | | | | | | |
| Photopolymerization | DAROCUR1173 (Ciba | — | — | 7.0 | 7.0 | 7.0 | 7.0 | 7.0 | 7.0 |
| initiator | Specialty Chemicals K.K.) | | | | | | | | |
| | IRGACURE379 (Ciba Specialty | — | — | 3.0 | 3.0 | 3.0 | 3.0 | 3.0 | 3.0 |
| | Chemicals K.K.) | | | | | | | | |

| | | | | | | |
|---|---|---|---|---|---|---|
| Total (% by mass) | 100.0 | 100.0 | 100.0 | 100.0 | 100.0 | 100.0 |

TABLE 2

| | | Viscosity | Molecular | | | |
|---|---|---|---|---|---|---|
| | | (mPa · s) | weight | Comp. Ex. 2 | Ex. 6 | Ex. 7 |
| Colorant | MOGUL-E | — | — | 7.0 | 7.0 | 7.0 |
| | (CABOT Corporation) | | | | | |
| Dispersant | SOLUSPHASE S33000 | — | — | 0.4 | 0.4 | 0.4 |
| | (Lubrizol Japan Ltd.) | | | | | |
| Extender pigment | Aerosil 200 | — | — | 3.5 | 3.5 | 3.0 |
| | (NIPPON AEROSIL CO., | | | | | |
| | LTD.) | | | | | |
| Monomer component | Monomer A | 7,000 | 578 | 5.0 | 23.0 | |
| (*) | Monomer B | 2,000 | 807 | | | |
| | Monomer C | 1,700 | 921 | | | |
| | Monomer D | 1,350 | 1,263 | | | 54.0 |
| | Monomer E | 1,500 | 1,947 | | | |
| | Monomer F | 150 | 427 | | | |
| | Monomer G | 110 | 540 | | | |
| | Monomer H | 250 | 768 | | 56.1 | 25.6 |
| | Monomer I | 610 | 776 | 74.1 | | |
| Photopolymerization | DAROCUR1173 (Ciba | — | — | 7.0 | 7.0 | 7.0 |
| initiator | Specialty Chemicals K.K.) | | | | | |
| | IRGACURE379 (Ciba | — | — | 3.0 | 3.0 | 3.0 |
| | Specialty Chemicals K.K.) | | | | | |

| | | | |
|---|---|---|---|
| Total (% by mass) | 100.0 | 100.0 | 100.0 |

TABLE 3

| | | Viscosity | Molecular | | | | | | |
|---|---|---|---|---|---|---|---|---|---|
| | | (mPa · s) | weight | Ex. 8 | Ex. 9 | Ex. 10 | Ex. 11 | Ex. 12 | Ex. 13 |
| Colorant | MOGUL-E (CABOT | — | — | 7.0 | 7.0 | 7.0 | 7.0 | 7.0 | 7.0 |
| | Corporation) | | | | | | | | |
| Dispersant | SOLUSPHASE S33000 | — | — | 0.4 | 0.4 | 0.4 | 0.4 | 0.4 | 0.4 |
| | (Lubrizol Japan Ltd.) | | | | | | | | |
| Extender pigment | Aerosil 200 | — | — | 3.0 | 3.0 | 3.5 | 2.5 | 2.5 | 3.0 |
| | (NIPPON AEROSIL CO., | | | | | | | | |
| | LTD.) | | | | | | | | |
| Monomer component (*) | Monomer A | 7,000 | 578 | | | | | | |
| | Monomer B | 2,000 | 807 | 52.7 | | | 52.5 | | |
| | Monomer C | 1,700 | 921 | | 56.0 | | | 52.5 | |
| | Monomer D | 1,350 | 1,263 | | | | | | |
| | Monomer E | 1,500 | 1,947 | | | 52.2 | | | 52.0 |
| | Monomer F | 150 | 427 | | | | | | |
| | Monomer G | 110 | 540 | 26.9 | 23.6 | 26.9 | | | |
| | Monomer H | 250 | 768 | | | | 27.6 | 27.6 | 27.6 |
| | Monomer I | 610 | 776 | | | | | | |
| Photopolymerization | DAROCUR1173 (Ciba | — | — | 7.0 | 7.0 | 7.0 | 7.0 | 7.0 | 7.0 |
| initiator | Specialty Chemicals K.K.) | | | | | | | | |
| | IRGACURE379 (Ciba Specialty | — | — | 3.0 | 3.0 | 3.0 | 3.0 | 3.0 | 3.0 |
| | Chemicals K.K.) | | | | | | | | |

| | | | | | | |
|---|---|---|---|---|---|---|
| Total (% by mass) | 100.0 | 100.0 | 100.0 | 100.0 | 100.0 | 100.0 |

TABLE 4

| | | Viscosity | Molecular | | | | | | Comp. |
|---|---|---|---|---|---|---|---|---|---|
| | | (mPa · s) | weight | Ex. 14 | Ex. 15 | Ex. 16 | Ex. 17 | Ex. 18 | Ex. 3 |
| Colorant | MOGUL-E (CABOT | — | — | 7.0 | 2.0 | 2.0 | 7.0 | 7.0 | 3.0 |
| | Corporation) | | | | | | | | |
| Dispersant | SOLUSPHASE S33000 | — | — | 0.4 | 0.3 | 0.3 | 0.4 | 0.4 | 0.3 |
| | (Lubrizo Japan Ltd.) | | | | | | | | |
| Extender pigment | Aerosil 200 (NIPPON | — | — | 4.0 | 2.0 | 5.0 | 4.0 | 3.0 | |
| | AEROSIL CO., Ltd.) | | | | | | | | |
| Monomer component (*) | Monomer A | 7,000 | 578 | 20.0 | | | | | 55.0 |
| | Monomer B | 2,000 | 807 | | | | | | |
| | Monomer C | 1,700 | 921 | | | | 28.0 | | |
| | Monomer D | 1,350 | 1,263 | 8.0 | 92.7 | 41.7 | | 60.0 | |
| | Monomer E | 1,500 | 1,947 | | | | | | |
| | Monomer F | 150 | 427 | 50.6 | | | 25.0 | | |
| | Monomer G | 110 | 540 | | | 41.0 | | | |
| | Monomer H | 250 | 768 | | | | | | |
| | Monomer I | 610 | 776 | | | | 25.6 | | 38.7 |
| | Monomer J | 85 | 698 | | | | | 19.6 | |
| Photopolymerization | DAROCUR1173 (Ciba | — | — | 7.0 | 3.0 | 7.0 | 7.0 | 7.0 | 3.0 |
| initiator | Specialty Chemicals K.K.) | | | | | | | | |
| | IRGACURE379 (Ciba | — | — | 3.0 | | 3.0 | 3.0 | 3.0 | |
| | Specialty Chemicals K.K.) | | | | | | | | |

| | | | | | | |
|---|---|---|---|---|---|---|
| Total (% by mass) | 100.0 | 100.0 | 100.0 | 100.0 | 100.0 | 100.0 |

(*) Details of the Monomers A to I, or monomer components, shown in Tables 1 to 4 are shown in Table 5.

TABLE 5

| | | | Number of | | | | |
|---|---|---|---|---|---|---|---|
| | | | functional | Molecular | Viscosity | | |
| Monomer | Structural formula | | groups | weight | (mPa · s) | Trade name | Manufactured by |
| Monomer A | Structural formula | m = 0, a = 0, b = 6 | 6 | 578 | 7,000 | KAYARAD DPHA | Nippon Kayaku Co., |
| | (2) | | | | | | Ltd. |
| Monomer B | Structural formula | m = 1, a = 2, b = 4 | 6 | 807 | 2,000 | KAYARAD DPCA- | Nippon Kayaku Co., |
| | (2) | | | | | 20 | Ltd. |
| Monomer C | Structural formula | m = 1, a = 3, b = 3 | 6 | 921 | 1,700 | KAYARAD DPCA- | Nippon Kayaku Co., |
| | (2) | | | | | 30 | Ltd. |
| Monomer D | Structural formula | m = 1, a = 6, b = 0 | 6 | 1,263 | 1,350 | KAYARAD DPCA- | Nippon Kayaku Co., |
| | (2) | | | | | 60 | Ltd. |
| Monomer E | Structural formula | m = 2, a = 6, b = 0 | 6 | 1,947 | 1,500 | KAYARAD DPCA- | Nippon Kayaku Co., |
| | (2) | | | | | 120 | Ltd. |
| Monomer F | Structural formula (5) | | 2 | 427 | 150 | KAYARAD R-526 | Nippon Kayaku Co., |
| | | | | | | | Ltd. |
| Monomer G | Structural formula | m + n = 2 | 2 | 540 | 110 | KAYARAD HX-220 | Nippon Kayaku Co., |
| | (3) | | | | | | Ltd. |
| Monomer H | Structural formula | m + n = 4 | 2 | 768 | 250 | KAYARAD HX-620 | Nippon Kayaku Co., |
| | (3) | | | | | | Ltd. |

| | | | | | | |
|---|---|---|---|---|---|---|
| Monomer I | Ethoxylated (10) bisphenol A | 2 | 776 | 610 | SR601 | Satomer |
| | diacrylate | | | | | |
| Monomer J | Polyethylene glycol diacrylate | 2 | 698 | 85 | New frontier | Dai-ichi Kogyo |
| | (EO: 13) | | | | PE-600 | Seiyaku Co., Ltd. |

In Table 5, “EO:13” means that the number of the ethylene oxide units is 13. Structural formulas (2), (3) and (5) are shown below. Additionally, “a”, “b”, “m” and “n” are average relative proportions of moieties of corresponding symbols in the following structural formulas. 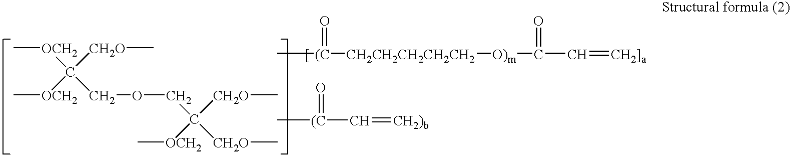 CH₂═(CHCO—(OCH₂CH₂CH₂CH₂CH₂CO)ₘ—OCH₂C(CH₃)₂—COO—OH₂—C(CH₃)₂—CH₂—O—(COCH₂CH₂CH₂CH₂CH₂O)ₙ—COCH═CH₂ Structural formula (3)
CH₂═CHCO—OCH₂—C(CH₃)₂—CH₂—OCO—(CH₂)₄—COO—CH₂—C(CH₃)₂—CH₂—O—COCH—CH₂ Structural Formula 5

The molecular weight and viscosity of each of the Monomers A to I were obtained as described below.

<Measurement Method of the Molecular Weights of Monomers A to I>

Structural formulas were identified for each monomer, and then, the molecular weight of each monomer was obtained based on the thus obtained structural formula.

<Measurement Method of the Viscosities of Monomers A to I>

The viscosity of each monomer was obtained using the Casson's equation. The measurement was conducted in the same manner as in the below-described measurement method for the viscosities of obtained monomer components, except that measurement temperature was set at 25° C.

<Evaluation>

As described below, the viscosity and average molecular weight of the monomer components were measured in each of the ultraviolet ray-curable inks of Examples 1 to 18 and Comparative Examples 1 to 3. And also, the ultraviolet ray-curable inks were evaluated for their curing properties and safety. The results are shown in Tables 6 to 9 for each ultraviolet ray-curable ink.

—Viscosity of Monomer Component—

Using CSR-10 (a stress rheometer manufactured by Bohlin Instruments), plastic viscosity was measured for each of the monomer components of the ultraviolet ray-curable inks of Examples 1 to 18 and Comparative Examples 1 to 3. More specifically, using a cone with a diameter of 2 cm and an angle of 2°, a flow curve was obtained under conditions—measurement temperature of 23° C. and stress continuously ranged from 12.5 Pa to 150 Pa—for each of the inks. Using the obtained flow curves, Casson plastic viscosities and Casson yield values were obtained with the following Casson's equation. Each value of the thus obtained Casson plastic viscosities was used as the viscosity of each monomer component.
\sqrt{τ}−\sqrt{τ₀}=\sqrt{Eta×D} Casson's Equation

where X represents a shear stress, τ0 represents a yield value, Eta represents a plastic viscosity and D represents a shear velocity.

<Curing Property of Ink>

Images were printed using the ultraviolet ray-curable inks of Examples 1 to 18 and Comparative Examples 1 to 3 in a screen printer (SATELIO A650, manufactured by Ricoh). Then, the recorded images were irradiated with ultraviolet rays using an ultraviolet ray irradiator containing three 400W medium pressure mercury lamps (HOK4/120, manufactured by Philips). Then, the printed images were rubbed with a piece of cloth being wet with water. The degrees of the smear of the printed images and cloth were visually evaluated using the following four evaluation criteria. In the evaluations, lower degree of smear means better curing property of the ink.

—Evaluation Criteria—

A: Smears were not recognized on both printed image and cloth.

B: Smears were not recognized on the printed image, while cloth was little stained.

C: Slight smears were recognized on both printed image and cloth.

D: Smears were recognized on both printed image and cloth.

<Measurement of the Average Molecular Weight of Monomer Component>

The average molecular weight of two monomers which were contained in the ultraviolet ray-curable ink for screen printing was calculated using the following equation, given that the molecular weight and relative proportion of one of the monomers were Mx and “a” respectively, and the molecular weight and relative proportion of the other monomer were My and “b” respectively.
Mxy=(aMx+bMy)/(a+b)

The average molecular weight of three monomers which were contained in the ultraviolet ray-curable ink for screen printing was calculated using the following equation, given that the molecular weight and relative proportion of any one of the monomers were Mx and “a” respectively, the molecular weight and relative proportion of another monomers were My and “b” respectively, and the molecular weight and relative proportion of the other monomer were Mz and “c” respectively.
Mxyz=(aMx+bMy+cMz)/(a+b+c)

<Evaluating Safety of Ink>

Using the following three criteria, evaluations in safety of each ink were conducted based on the obtained average molecular weights of the monomer components.

—Evaluation Criteria—

A: the molecular weight was 800 or more, and the ink was barely irritable to the skin.

B: the molecular weight was in the range of 600 or more to less than 800. The ink was slightly irritable, while it was acceptable in the practical use.

C: the molecular weight was in the range of 500 or more to less than 600. The ink was irritable to the skin, while it was acceptable in the practical use.

D: the molecular weight was less than 500. The ink was irritable to the skin, and it cannot be used in the practical use.

<Evaluating 60° C. Stability>

The inks were kept under 60° C. for 2 weeks. Then, the viscosity of each ink was measured to compare with that of the ink before kept for 2 weeks.

A: Slightly change was recognized in the property of the ink.

B: Change was recognized in the property of the ink, while it was acceptable in the practical use.

C: Substantial change was recognized in the property of the ink. It was not acceptable in the practical use.

TABLE 6

| | Comp. | | | | | |
|---|---|---|---|---|---|---|
| | Ex. 1 | Ex. 1 | Ex. 2 | Ex. 3 | Ex. 4 | Ex. 5 |
| Viscosity of monomer | 462 | 491 | 456 | 446 | 457 | 493 |
| component (mPa · s) | | | | | | |
| Curing property of ink | C | B | B | B | B | A |
| Average molecular | 471.2 | 601.0 | 653.3 | 841.8 | 1161.9 | 972.3 |
| weight of monomer | | | | | | |
| component | | | | | | |
| Safety | D | B | B | A | A | A |
| 60° C. Stability | B | B | B | B | B | B |

TABLE 7

| | Comp. | | |
|---|---|---|---|
| | Ex. 2 | Ex. 6 | Ex. 7 |
| Viscosity of monomer | 712 | 659 | 785 |
| component (mPa · s) | | | |
| Curing property of ink | D | C | A |
| Average molecular | 763.5 | 712.8 | 1103.8 |
| weight of monomer | | | |
| component | | | |
| Safety | B | B | A |
| 60° C. Stability | B | B | B |

TABLE 8

| | Ex. 8 | Ex. 9 | Ex. 10 | Ex. 11 | Ex. 12 | Ex. 13 |
|---|---|---|---|---|---|---|
| Viscosity of | 750 | 755 | 670 | 977 | 878 | 806 |
| monomer | | | | | | |
| component | | | | | | |
| (mPa · s) | | | | | | |
| Curing property | A | A | A | A | A | A |
| of ink | | | | | | |
| Average molecular | 716.8 | 808.0 | 1468.5 | 793.6 | 868.3 | 1538.2 |
| weight of monomer | | | | | | |
| component | | | | | | |
| Safety | B | A | A | B | A | A |
| 60° C. Stability | B | B | B | B | B | B |

TABLE 9

| | | | | | | Comp. |
|---|---|---|---|---|---|---|
| | Ex. 14 | Ex. 15 | Ex. 16 | Ex. 17 | Ex. 18 | Ex. 3 |
| Viscosity of | 499 | 1350 | 389 | 562 | 740 | 2555 |
| monomer | | | | | | |
| component | | | | | | |
| (mPa · s) | | | | | | |
| Curing | C | A | A | C | A | A |
| property of ink | | | | | | |
| Average | 550.5 | 1263.0 | 904.6 | 716.6 | 1123.9 | 659.8 |
| molecular | | | | | | |
| weight of | | | | | | |
| monomer | | | | | | |
| component | | | | | | |
| Safety | C | A | A | B | A | B |
| 60° C. | B | B | B | B | A | B |
| Stability | | | | | | |

As shown in Tables 6 to 9, the ultraviolet ray-curable inks of Examples 1 to 18, which respectively contain at least one monomer component having the unit represented by the structural formula (1) in its molecule, had excellent curing properties and safety, low viscosities and low molecular weights of the monomer components compared with the ultraviolet ray-curable inks of Comparative Examples 1 to 3. And further, it was found that the ultraviolet ray-curable ink of Example 18, which has an ethylene oxide unit represented by the structural formula (4), can improve the flowability and stability (60° C. stability) of the ink that was kept under a high temperature.

### INDUSTRIAL APPLICABILITY

The ultraviolet ray-curable ink for screen printing that is one of the embodiments of the present invention has an excellent curing property and in which the molecular weight of monomer components which are related to safety issues can be increased without increasing the viscosity of the ink. Thus, the ultraviolet ray-curable ink can be suitably used for screen printing using a rotary screen printing machine.

What is claimed is:

1. An active energy beam-curable ink, comprising:
active energy beam-curable monomer components,
wherein at least one of the active energy beam-curable monomer components contains a unit represented by the following structural formula (1) in its molecule.
—(CO—CH2CH2CH2CH2CH2—O)— Structural formula (1)

2. The active energy beam-curable ink according to claim 1, wherein at least one active energy beam-curable monomer component having the unit represented by the structural formula (1) in its molecule is di-pentaerythritol hexaacrylate modified with ε-caprolactone and is represented by the following structural formula (2). 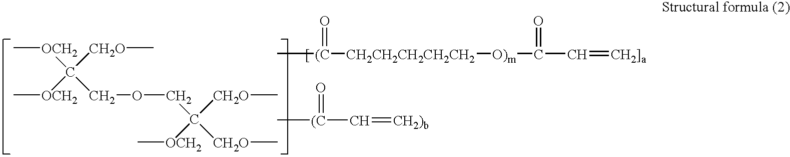
Where “a”, “b” and “m” are average values, “a” is an integer in the range of 1 to 6, “b” is an integer in the range of 0 to 6, a+b=6, and “m” is an integer in the range of 1 to 2.

3. The active energy beam-curable ink according to claim 2, wherein “a” represents 6 and “b” represents 0 in the structural formula (2).

4. The active energy beam-curable ink according to claim 1, wherein at least one active energy beam-curable monomer component having the unit represented by the structural formula (1) in its molecule is hydroxypivalate neopentylglycol ester diacrylate modified with ε-caprolactone and is represented by the following structural formula (3).
CH₂═(CHCO—(OCH₂CH₂CH₂CH₂CH₂CO)ₘ—OCH₂C(CH₃)₂—COO—OH₂—C(CH₃)₂—CH₂—O—(COCH₂CH₂CH₂CH₂CH₂O)ₙ—COCH═CH₂ Structural formula (3)
Where “a”, “b” and “m” are average values, “m” is an integer in the range of 0 to 4, “n” is an integer in the range of 0 to 4, and m+n=2 to 4.

5. The active energy beam-curable ink according to claim 1, wherein all of the active energy beam-curable monomer components have the unit represented by the structural formula (1) in their molecules.

6. The active energy beam-curable ink according to claim 1, which is used for screen printing.

7. The active energy beam-curable ink according to claim 1, further comprising a monomer having ethyleneoxide (EO) units represented by the structural formula (4).
—(CH2CH₂O)— Structural formula (4)

8. The active energy beam-curable ink according to claim 7, wherein the number of the ethyleneoxide (EO) units represented by the structural formula (4) is in the range of 6 to 14.

9. The active energy beam-curable ink according to claim 1, further comprising a silica.
